(19) United States
(12) Patent Application Publication
(10) Pub. No.: US 2022/0236204 A1
(43) Pub. Date: Jul. 28, 2022

(54) TEMPERATURE-CONTROL DEVICE AND METHOD FOR A FLASH-POINT DETERMINATION TEST AND/OR FIRE-POINT DETERMINATION TEST

(71) Applicant: ANTON PAAR PROVETEC GMBH, Blankenfelde-Mahlow (DE)

(72) Inventors: Christian Andreas HEINE, Berlin (DE); Robert SKOLE, Berlin (DE); Martin HETTEGGER, Schwarzach (AT); Florian STRASSER, Berlin (DE); Xenia ERLER, Berlin (DE)

(21) Appl. No.: 17/596,188

(22) PCT Filed: Apr. 24, 2020
(86) PCT No.: PCT/EP2020/061540

§ 371 Date: Dec. 3, 2021

(30) Foreign Application Priority Data
Jun. 5, 2019 (DE) 10 2019 115 120.1

(51) Int. Cl. G01N 25/52; G01N 33/28; G05D 23/185

(52) U.S. Cl. CPC G01N 25/52; G05D 23/185; G01N 33/28

(57) ABSTRACT

A device for tempering a sample located in a container for a flash point determination test and/or fire point determination test is provided, the device comprising: a temperature control block having a, in particular cylindrical, container receptacle for receiving the container; a cooling air guide body for delimiting a cooling air path in which the temperature control block is arranged; wherein the temperature control block has an outer surface with fins.

This application is the U.S. national phase of International Application No. PCT/EP2020/061540 filed 24 Apr. 2020 which designated the U.S. and claims priority to German Patent Application No. 10 2019 115 120.1 filed 5 Jun. 2019, the entire contents of each of which are hereby incorporated herein by reference.

### FIELD OF THE INVENTION

Embodiments of the present invention relate to a device as well as a method for tempering a sample located in a container for a flash point determination test and/or a fire point determination test. Furthermore, the present invention relates to a flash point determination apparatus, which is in particular also designed for fire point determination, comprising the temperature control device.

### BACKGROUND

Flash point test equipment is conventionally used to characterize fuels (e.g. diesel, gasoline, kerosene, fuel oil), solvents, lubricating oils or chemicals. By definition, the flash point is the lowest temperature at which vapors (gaseous sample mixed with air) evolve in an open or closed vessel or crucible from the liquid to be tested under specified conditions in such quantity that a sample gas-air mixture flammable by external ignition is formed inside or outside the container.

To determine the flash point and/or the fire point (burning point), preferably according to various standards, a defined quantity of a sample (substance) to be examined is filled into the container (e.g. measuring crucible), heated in a controlled manner (in particular brought to a predetermined temperature) and stirred as required. During this process, a gaseous phase continuously forms above the liquid sample. At a certain temperature, an ignition source is introduced into the container at periodic time and/or temperature intervals to ignite the formed gas-air sample mixture. If a flame is detected at a certain sample temperature whose burning time is less than 5 seconds, the flash point is determined. If the burning time is longer than 5 seconds, the fire point of the sample is determined.

Various standard methods are suitable for flash point determination, which are essentially characterized by the methods according to i) Pensky, ii) Pensky-Martens, iii) Abel, iv) Abel-Pensky, v) Tagliabue and vi) Cleveland.

Document CN 101839877 B discloses a flash point test system, wherein an external cooling is provided to reduce the temperature of the flow medium. The flash point or fire point test device is connected to the external cooling device via a tube.

Document CN 205920076 U discloses a fully automatic test device suitable for gas auto-ignition temperature determination, wherein a heating system with temperature control is provided.

Document CN 202075255 U discloses a semi-automatic flash point test system for petroleum products, wherein a heater is mounted in the lower housing.

Document JP 4287314 B2 discloses an apparatus for measuring a flash point, wherein a heat transfer medium is cooled by a cooler.

Document JP 560119453 A discloses a flash point determination measuring apparatus, wherein a liquid sample is heated by a heater to vaporize the liquid. The flash time can be detected by detecting the change in sound or light.

A conventional flash point test device may have a heating assembly which serves to regulate and control the sample temperature. The heating rate of the sample is defined by the standard only within a certain temperature range.

Outside a temperature range defined by the standard, the heating and cooling rate can be freely selected. The design of the heating/cooling assembly determines the maximum sample throughput. The sample throughput of a flash point tester or fire point tester is mainly composed of three temperature rates: i) heating rate up to the temperature range relevant to the standard, ii) heating rate prescribed in the standard in the temperature range relevant to the standard and iii) cooling rate after completion of the flash point determination or fire point determination. While the heating rate prescribed in a certain temperature range according to the standard is invariable, however, the heating rate up to the range relevant to the standard as well as the cooling rate after completion of the flash point determination or fire point determination can be freely selected and can thus influence the overall duration of the experiment. The parameters i) and iii) not specified by a standard directly result from the technical design of the heating/cooling assembly.

In conventional devices for fire point determination or flash point determination, the required time durations of the experiment are relatively long, so that the sample throughput is relatively low.

Thus, there may be a need to provide a device or a method for tempering a sample located in a container for a flash point determination test and/or a fire point determination test, wherein experimental limitations defined by a standard can be complied with, but an overall experiment duration may be reduced or the sample throughput may be increased. In addition, there may be a need to provide an improved heating/cooling assembly that conforms to a standard, whereby heating or cooling may be achieved as rapidly as possible in the temperature ranges not controlled by the standard(s). Thus, the overall process time may be significantly reduced and the sample throughput may be significantly increased.

### SUMMARY OF THE INVENTION

This need may be met by the subject matter of the independent claims. The dependent claims specify particular embodiments of the present invention.

According to an embodiment of the present invention, there is provided a device for tempering (controlling temperature of) a sample located (contained) in a container for a flash point determination test and/or a fire point determination test, the device comprising: a temperature control block (tempering block) having a container receptacle, in particular a cylindrical container receptacle, for receiving the container; a cooling air guide body for delimiting a cooling air path in which the temperature control block (for air cooling) is arranged; wherein the temperature control block has an outer surface with fins (e.g., ribs, ridges, splines, protrusions, projections, bulges, overhangs, lamellae with intervening depressions, channels, grooves or furrows).

The device for tempering may be suitable for a standardized flash point determination test and/or fire point determination test which, for example, correspond to or comply with one or more of the following standards (in each case at least for the versions valid on the filing date): ASTM D93, DIN EN ISO 2719, GB/T261, IP 34, JIS K 2265, ISO 13736, ISO 1516, ISO 1523, DIN 51755-1 (Abel-Pensky with corresponding equipment); ASTM D56, ASTM D3934, ASTM D3941; ASTM D92, DIN EN ISO 2592, IP 36, IP 403. Embodiments may comply with further standards not listed herein. Embodiments of the present invention supported one or more of the methods according to i) Pensky and/or ii) Pensky-Martens and/or iii) Abel and/or iv Abel-Pensky and/or v) Tagliabue and/or vi) Cleveland.

Embodiments of the present invention may in particular employ the methods according to H) Pensky-Martens, ii) Cleveland. In this regard, the devices or apparatus may comply with the following standards: ASTM D93, EN ISO 2719, GB/T261, IP 34, JIS K2265; ASTM D92, EN ISO 2592, IP 36, IP 403, JIS K2265 (in each case at least for the versions valid on the filing date).

According to embodiments of the present invention, an advantageous design of the heating/cooling assembly allows for an increased sample throughput.

The flash point determination test and/or fire point determination test may be used e.g. for kerosene, oil, substances containing hydrocarbons in general, e.g. for quality testing. The flash point and/or fire point test may be carried out, for example, with one of the test setups developed by Sir Frederik Abel, Adolf Martens, Berthold Pensky or Charles J. Tagliabue.

During the flash point determination test and/or fire point determination test, the sample to be tested may be contained in a closed container or in an open container. Both classes of flash point tests are supported by embodiments of the present invention. Embodiments of the present invention support test methods wherein an equilibrium state, a non-equilibrium state, or a fast equilibrium state may be present within the container. Non-equilibrium state methods may comply with, for example, one or more of DIN EN ISO 13736, ASTM D56, DIN EN ISO 2719, ASTM D93, DIN EN ISO 2592, ASTM D92. Equilibrium state methods may comply with, for example, one or more of the standards DIN EN ISO 1516, DIN EN ISO 1523, DIN EN 924, ASTM D3941, DIN 53213. Fast equilibrium state methods may comply with, for example, the standard DIN EN ISO 3679.

While performing the flash point determination test, the sample to be tested may be stirred. While performing the flash point determination test, the temperature of the sample within the container may be measured at one or more locations (such as in the gas phase and/or the liquid phase). Further, the atmospheric pressure and/or the pressure within the container may be measured and the measurement results may be corrected accordingly. The flash point determination apparatus according to embodiments of the present invention may, for example, be configured to determine flash points in a range of −40° C. to +410° C.

In particular, the container may be a substantially cylindrical container with a lid or without a lid.

For example, the container may be substantially cylindrical. In the liquid state, the sample may fill, for example, about ⅓ to ⅔ of the interior of the container. Above the liquid level of the sample within the container, the sample may be present in a gaseous state, in particular mixed with air.

The device for tempering the sample may be configured to heat and/or cool the sample. The sample may be a liquid sample, which may partially also be in a gaseous state within the container.

The temperature control block may be made of metal. The container receptacle (e.g. a, in particular cylindrical, recess in the temperature control block) may surround the container laterally as well as below. The container may, for example, have a lateral and lower outer surface directly or immediately adjacent to or in contact with a lateral and lower (inner) surface of the container receptacle. This allows for good thermal conduction between the temperature control block and the container.

To heat the sample located in the container, the temperature control block may be heated, for example with an electric heating wire, and transfer heat to the container by thermal radiation, by thermal conduction or diffusion, and/or by convection. The container may then transfer the heat to the sample located in the container.

In a cooling process, the heat flow is in the opposite direction, i.e. from the sample located in the container to the container and from the container to the temperature control block.

The cooling air guide body may be made of metal and may determine the direction of movement of cooling air based on its geometry. Cooling air may flow within the cooling air guide body with a flow direction that is substantially determined by the geometry of the cooling air guide body.

The surface of the temperature control block may have an inner surface and the outer surface. The inner surface and/or the outer surface may be suitably treated, coated or the like. The inner surface of the temperature control block may define the container receptacle, and the remainder of the surface may form the outer surface. The outer surface of the temperature control block is understood to be that portion which does not define the container receptacle for receiving the container. A portion of the outer surface or the entire outer surface of the temperature control block may comprise fins. The inner surface of the temperature control block may be substantially smooth to allow as direct contact as possible or a defined distance with the container, which may also have a smooth outer surface. If the outer surface of the temperature control block is provided with fins, a heat exchange with cooling air flowing around the temperature control block or the outer surface may be improved. In particular, an area size of the outer surface is larger due to the fins than if the outer surface would not have fins, for example would be smooth. Due to the fins or the increased area size of the outer surface, a cooling rate may be increased compared to conventional systems. Thus, for example, a sample may be cooled down again more quickly after determination of the flash point or the fire point, so that it may be manipulated without danger in order to be able to carry out a further test with a further sample.

The fins may be understood as elongate protrusions, such as projections, bulges, protrusions and/or lamellae, between each of which a channel or furrow is formed. The fins may be formed, for example, due to different wall thicknesses of the temperature control block. A minimum wall thickness may be present, for example, in a region between two fins and may be, for example, between 1 mm and 10 mm. A maximum thickness may be present, for example, at the positions of the fins and may be, for example, between 6 mm and 30 mm. In cross-section, the fins (at least first fins) may have the same or different shapes, for example a trapezoidal shape or wave shape or sawtooth shape or rectangular shape or the shape of a polygon. Second fins (e.g. on a lower outer surface of the temperature control block) may have the same or different shapes in cross-section, e.g. a rectangular shape.

For producing the fins, parts of the outer surface of the temperature control block may, for example, be milled out or turned out, wherein the fins are formed between the depressions created by the milling out or turning out. The furrows or channels formed between the fins may have, for example, a width decreasing radially inwardly, in particular those furrows or channels formed between fins which are formed on lateral outer surfaces of the temperature control block. The furrows or channels between the fins may have, for example, chamfers which form sloping flanks of the fins. At a lower outer surface region of the temperature control block, the flanks of the fins may form parallel surfaces. The flanks of the fins may be substantially planar or form part of a conical surface, in particular form an annular part of a conical surface. The fins may be formed in different geometries.

The temperature control block may have a substantially cylindrical symmetry, at least notwithstanding a lower portion of the temperature control block. The fins may be circumferentially formed in the circumferential direction and may also obey the cylindrical symmetry. In a cross-sectional view, the lateral fins (i.e., the fins provided at a side outer surface) may resemble a gear rack, with raised portions alternating with recessed portions. The lateral (first) fins may all be formed substantially the same, i.e. having the same geometry and dimensions in terms of fin height, for example, and groove depth or channel depth. In contrast, the lower (second) fins may have different dimensions, e.g., fins having different fin heights or different channel depths or groove depths therebetween.

The cooling air path is the free space delimited by the cooling air guide body in which cooling air may flow, in particular towards and around the temperature control block. Thus, during a cooling process, the temperature control block is exposed to a cooling air flow within the cooling air path to be able to cool the temperature control block. At least the outer surface of the temperature control block is exposed to cooling air within the cooling air path. Thus, the cooling air comes into contact with the fins within the cooling air path, and in particular may flow in channels or furrows formed between the fins, wherein the cooling air is in direct contact with the flanks and top edges or surfaces of the fins, and the valleys (or grounds or bottoms) between the fins. Here, “top” refers to the radially outermost region, while terms such as “lower” or “bottom” refer to the radially innermost region.

According to an embodiment of the present invention, a cooling channel is formed between each two adjacent fins, within which cooling air flows substantially parallel to the fins. The cooling channel (between each two adjacent fins) may thus be delimited by a flank of a first fin and a flank of a second fin adjacent to the first fin, as well as by a bottom (e.g. lowest or radially innermost point or region) between the two fins. In particular, the cooling channel may be formed circumferentially in the circumferential direction around the lateral outer surface of the temperature control block. Within the cooling channel, the cooling air may flow with low turbulence and in particular with less stalling. The cooling air may flow substantially along the longitudinal extension direction of the cooling channels.

According to an embodiment of the present invention, the temperature control block has a greater wall thickness at positions of fins than at positions between fins. The temperature control block may thus have varying wall thickness. In particular, in an upper region, the side wall of the temperature control block may have a wall thickness varying in a vertical direction. In this regard, at positions of upper edges of fins, the wall thickness may be maximum and at a bottom (or floor or valley) exactly in the middle between two adjacent fins, the wall thickness may be minimum. Due to the fins, the outer surface, in particular lateral outer surface, of the temperature control block may be designed as corrugated, while the inner surface of the temperature control block (which is in contact with the container) may be designed as smooth. The different wall thicknesses may be formed by milling or turning out material, whereby furrows or channels may be formed between which the fins remain.

According to an embodiment of the present invention, the cooling air guided in the cooling air guide body has a substantially horizontal flow direction in the region of the temperature control block. Here, the directional designations horizontal and vertical are to be understood with reference to a use of the temperature control device during a flash point determination test and a fire point determination test, respectively. During such a test, the temperature control block is oriented such that a cylinder symmetry axis is along the vertical direction. The horizontal direction or horizontal plane is perpendicular to the vertical direction. The cylinder symmetry axis may also be inclined relative to the vertical by a certain angle, for example 2°, 5° or 10°. Then the directional designation horizontal means a direction orthogonal to the cylinder symmetry axis. When the cooling air has a substantially horizontal flow direction, the temperature control block may be effectively cooled, in particular uniformly from all sides of the temperature control block. Further, the lower outer surface of the temperature control block may be effectively cooled. In particular, the cooling air may have a flow direction within the cooling air path in the region of the temperature control block which has only minor or small or evanescent components in the vertical direction. Thus, the cooling air may have only minor flow components in directions transverse to the fins or the cooling channels. Cooling air flowing in the cooling channels may thus effectively contribute to cooling the temperature control block.

According to an embodiment of the present invention, the outer surface of the temperature control block comprises a shell surface (lateral surface) and a lower outer surface (bottom outer surface), wherein the shell surface and/or the lower outer surface are exposed to cooling air within the cooling air guide body. If both the shell surface and the lower outer surface are exposed to the cooling air, a cooling rate may be further increased. Further, it is advantageous if substantially the entire lateral outer surface within the cooling air path is exposed to the cooling air, in particular at least 80% or at least 90% or at least 95% of the lateral outer surface of the temperature control block. The shell surface may have cylindrical symmetry and may form a circumferential lateral outer surface. The lower outer surface may be substantially circular, for example, in a view along the vertical direction. According to other embodiments of the present invention, the lower outer surface may be elliptical or polygonal.

According to an embodiment of the present invention, first fins are each formed in a circular circumferential manner and form parts of the shell surface of the temperature control block. When the first fins are formed circularly circumferentially, they may be readily fabricated, for example, by milling or turning out material at positions between fins to be formed. Each fin may extend, for example, radially outwardly as well as circumferentially (e.g., in a horizontal plane). Each fin may have, for example, an upper surface (e.g., at a most radially outwardly projecting level) and two edge surfaces or flanks extending away from the upper surface. The area (e.g., at a least radially outwardly projecting level) between two fins is also referred to as a bottom of a furrow or channel between the fins. According to other embodiments of the present invention, the first fins may each be formed in an elliptical or polygonal circumferential shape.

According to an embodiment of the present invention, the first fins, in particular circular fins, extend parallel to each other in different horizontal planes vertically spaced apart from each other. The orientation of the fins is thus adapted or matched to the geometry of the cooling air guide body in that the flow of cooling air in a substantially horizontal direction corresponds to the orientation of the fins, so that the cooling air flows laterally around the lateral outer surface of the temperature control block in different horizontal planes along the cooling channels between the fins.

According to an embodiment of the present invention, the device is configured in such a way that a first, in particular circular, cooling channel is formed between each two adjacent first fins, within which cooling air flows in the circumferential direction of the temperature control block in a clockwise direction in one part of the cooling channel and in a counterclockwise direction in another opposite part of the cooling channel. The cooling air may thus be guided (directed) around the side surfaces of the temperature control block in two parts, a first part in a clockwise direction and a second part in a counterclockwise direction. Each circular cooling channel may lie in an associated horizontal plane. This allows a flow with few flow separations (or stallings) around the temperature control block, which may lead to an effective cooling.

According to an embodiment of the present invention, second fins are provided at the lower surface (e.g., base surface or face surface) of the temperature control block. The second fins may thus further contribute to an effective cooling, as the lower outer surface also has a larger surface area compared to a completely smooth outer surface, which increases a heat exchange rate.

According to an embodiment of the present invention, the device is configured such that the second fins extend parallel to each other in a horizontal plane and are laterally spaced apart from each other in a horizontal direction perpendicular to the flow direction of the cooling air, wherein a second, in particular rectilinear, cooling channel is formed between each two adjacent second fins, within which cooling air flows.

Also in the second cooling channel or in each second cooling channel, the cooling air may flow substantially in a horizontal direction and in particular in a flow direction in a horizontal plane which substantially corresponds or is similar to an inflow direction which is also predetermined by the geometry of the cooling air guide body.

According to an embodiment of the present invention, at least one thermal protection element (heat protection element) is arranged within the cooling air guide body upstream of the temperature control block, which absorbs parts of a thermal radiation originating from the temperature control block and/or reduces a convection of air from the temperature control block to another component. In particular, a plurality of thermal protection elements may be provided, in particular two thermal protection elements arranged at different vertical positions, During a flash point determination test or fire point determination test, the temperature control block may be heated to relatively high temperatures, which may risk damaging components of the device or a flash point determination apparatus or fire point determination apparatus. To protect further components from damage due to heat exposure, the at least one thermal protection element is provided, which may be made of metal to effectively shield absorbed heat. The thermal protection element may be formed as a movable element to be able to support different stages of measurement during a flash point determination test or fire point determination test. For example, the thermal shield element may be in different orientations or states at different stages of the measurement.

According to an embodiment of the present invention, the thermal protection element comprises at least one pivotable thermal protection flap (thermal damper), wherein the thermal protection flap in the open state, in particular in a substantially horizontal position, substantially clears the cooling air path and in the closed state, in particular vertical position, at least partially blocks the cooling air path.

The thermal protection flap may be formed as a substantially planar member or as a planar plate, wherein a pivot axis may lie in the horizontal plane. In particular, a pivot axis may lie in a horizontal plane and perpendicular to an inflow direction of the cooling air. Thus, the cooling air path may be advantageously cleared (unblocked) when the thermal protection flap is in the open state and blocked when the thermal protection flap is in the closed state. If a plurality of thermal protection flaps is provided, they may be arranged vertically adjacent to each other, for example. Depending on the size of the cooling air path, one or more thermal protection flaps may be provided.

According to an embodiment of the present invention, the at least one thermal protection flap transitions (changes) from the closed state to the open state by pivoting due to a cooling air flow during a cooling operation. Thus, an additional actuator for actively moving the at least one thermal protection flap may be dispensed with, since the at least one thermal protection flap transitions from the closed state to the open state solely due to the cooling air flow. In other embodiments, an additional actuator may be provided to transfer the at least one thermal protection flap to the open state and/or the closed state.

According to an embodiment of the present invention, a cross-sectional size of the cooling air path decreases in the region of the temperature control block from upstream to downstream. The terms upstream and downstream, respectively, refer to relative positions along the cooling air flow path. The temperature control block may have cooling air flowing into it from an upstream side (inflow side), and the cooling air may exit the temperature control block at a downstream side (outflow side). The upstream side of the temperature control block is thus upstream and the downstream side is downstream in a relative observation. At the upstream side, the cooling air has a lower temperature than at the downstream side, thus has a more effective cooling effect at the upstream side than at the downstream side. In order to increase the flow velocity at the downstream side, at which the cooling air already has an increased temperature, a reduction of the cross-sectional size of the cooling air path towards the downstream side is provided. By doing so, an increase in the cooling effect of the already heated cooling air may be achieved. The geometry of the cooling air path, and thus the geometry of the cooling air guide body, may be determined according to simulations, which may thus also be used to optimize the cross-sectional size at different locations within the air cooling path to achieve an optimized cooling air. For example, the cross-sectional area at the downstream side is less than 90%, in particular less than 80% or less than 70% of the cross-sectional area at the upstream side.

According to an embodiment of the present invention, the cooling air guide body comprises an inlet opening for admitting cooling air from outside the device, wherein the device further comprises a fan, in particular a radial fan (radial ventilator, centrifugal fan), upstream of the temperature control block and/or the thermal protection element, which is configured to convey the cooling air admitted via the inlet opening from the outside to the inside of the cooling air guide body towards the temperature control block.

The cooling air may thus comprise ambient air. In other embodiments, cooling air may comprise pre-cooled air (by means of a further component). The inlet opening may comprise, for example, a grid or grate behind which the fan is provided. Instead of a radial fan, an axial fan may also be used. Also, multiple fans may be used. The fan may, for example, be arranged vertically below a lower outer surface of the temperature control block.

According to an embodiment of the present invention, the cooling air guide body is formed such that the cooling air (within the cooling air guide body) flows to the temperature control block at an upstream side with an inflow direction, flows around the temperature control block laterally and/or underneath, and leaves the temperature control block at a downstream side opposite the upstream side with an outflow direction, wherein the outflow direction is substantially equal to the inflow direction. If the outflow direction is substantially equal to the inflow direction, the cooling air may flow around the outer surfaces of the temperature control block with substantially few flow separations to thereby improve the cooling effect.

According to an embodiment of the present invention, the device further comprises a temperature sensor configured to measure the temperature of the temperature control block and arranged in particular centrally at a lower end wall of the temperature control block. A temperature sensor may be used to control the temperature. A central arrangement may allow a reliable temperature measurement.

According to an embodiment of the present invention, the temperature control block comprises an electric heating wire for heating the temperature control block, which is arranged in particular within the lower end wall of the temperature control block, further in particular circumferentially in the circumferential direction. Within the lower end wall, the temperature control block may have a greatest wall thickness. If the heating wire is arranged circumferentially in the circumferential direction, uniform heating of the temperature control block and thus also of the sample container may be achieved.

According to an embodiment of the present invention, the device further comprises a controller configured to control the fan and/or the heating wire depending on the measured temperature of the temperature control block. By controlling at least the fan, the cooling rate may be adjusted, and by controlling at least the heating wire, the heating rate may be controlled.

According to an embodiment of the present invention, a flash point determination apparatus is provided, in particular also adapted for fire point determination, the flash point determination apparatus comprising: a container for receiving a sample to be tested; a device for tempering the sample located (contained) in the container according to any one of the preceding claims, the container being insertable into the container receptacle of the temperature control block; and an ignition device for igniting the sample.

It should be understood that features which have been described, referred to, explained or provided, individually or in any combination, in connection with a device for tempering a sample located in a container for a flash point determination test and/or a fire point determination test may also be applied, individually or in any combination, to a method of tempering a sample located in a container for a flash point determination test and/or a fire point determination test, and vice versa, in accordance with embodiments of the present invention.

According to an embodiment of the present invention, a method of tempering a sample located in a container for a flash point determination test and/or a fire point determination test is provided, the method comprising: receiving the container in a, in particular cylindrical, container receptacle of a temperature control block; cooling an outer surface of the temperature control block having fins within a cooling air path delimited by a cooling air guide body.

Further advantages and features of the present invention will be apparent from the following exemplary description of embodiments. The invention is not limited to the embodiments described or illustrated.

### BRIEF DESCRIPTION OF THE DRAWINGS

FIG. 1 illustrates, in a schematic sectional view, a flash point determination apparatus, in particular also designed for fire point determination, according to an embodiment of the present invention;

FIG. 2 illustrates, in a schematic perspective sectional view, a device for tempering a sample located in a container according to an embodiment of the present invention;

FIGS. 3A, 3B, and 3C illustrate, in a sectional view, a perspective view, and a cross-sectional perspective view, respectively, a temperature control block as it may be provided in a device for tempering a sample according to an embodiment of the present invention; and

FIG. 4 illustrates, in a schematic sectional illustration with a viewing direction along the vertical direction, a cooling air flow as it may be generated in embodiments of the present invention.

### DETAILED DESCRIPTION OF AN EMBODIMENT

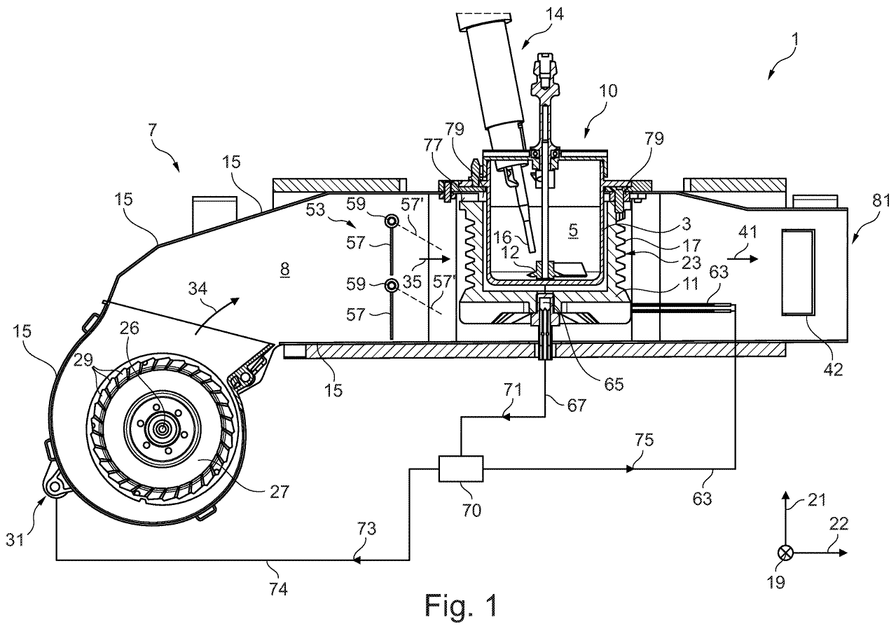

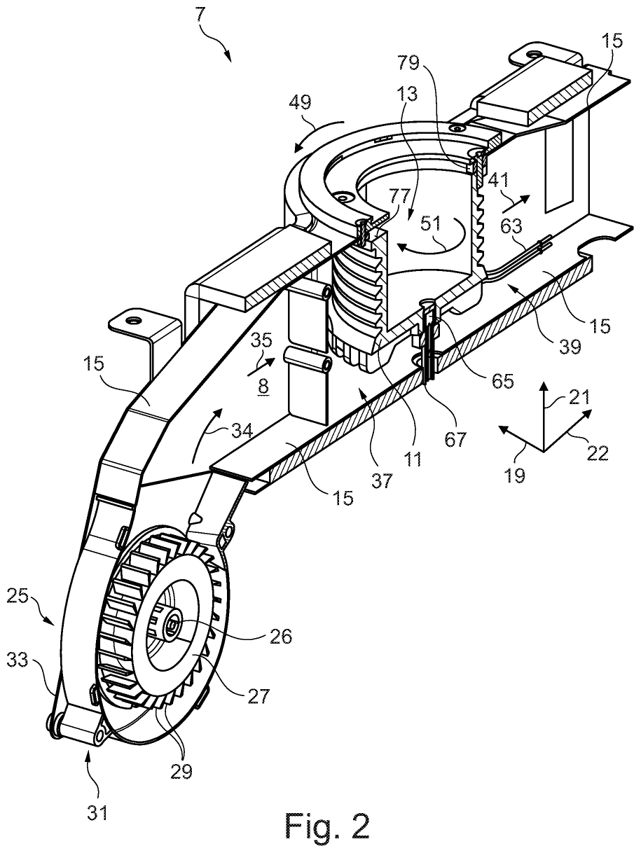

According to an embodiment of the present invention, the flash point determination apparatus 1 shown in FIG. 1 in a sectional view, which is in particular also designed for fire point determination, comprises a container 3 for receiving a sample 5 to be examined, which is in a liquid state. Furthermore, the flash point determination apparatus 1 comprises a device 7 for tempering the sample 5 locared in the container according to an embodiment of the present invention, which is also illustrated in a perspective sectional view in FIG. 2. The flash point determination apparatus 1 further comprises an ignition device not shown, which is provided for igniting the sample 5 within the container 3, a stirring device 10 with stirrer 12, and a flash point and temperature detector 14 with temperature sensor 16, which extends into the liquid part of the sample 5.

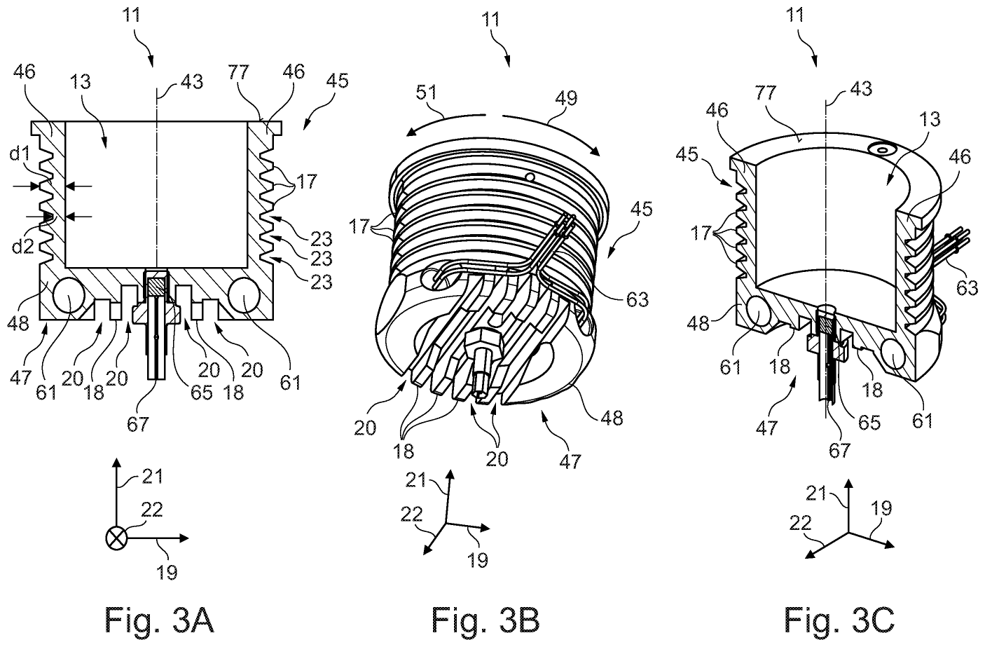

According to an embodiment of the present invention, the device 7 for tempering the sample 5 located in the container 3 for a flash point determination test and/or a fire point determination test comprises a temperature control block 11 as also illustrated in FIGS. 3A, 3B, 3C, with a, in particular cylindrical, container receptacle 13 for receiving the container 3. The device 7 further comprises a cooling air guide body 15 for delimiting a cooling air path 8 in which the temperature control block 11 is arranged for air cooling.

In this regard, the temperature control block 11 has an outer surface with fins 17, 18. As seen in the sectional view in FIG. 3A along a horizontal direction 19, a cooling channel 23 is formed between each two adjacent first fins 17, within which cooling air flows substantially parallel to the fins 17. As can also be seen from FIG. 3A, the temperature control block 11 has a wall thickness d1 at positions of the first fins 17 which is greater than the wall thickness d2 at positions between the first fins 17. The depth of the channels or height (radial extent) of the fins 17 may be, for example, between 5 mm and 30 mm. The (vertical) distance between two of the fins 17 may be, for example, between 2 mm and 15 mm.

The device 7, and in particular the cooling air guide body 15, further comprises an inlet opening 25 for admitting cooling air 34 from outside the device, and the device 7 further comprises a fan 27, in particular a radial fan, upstream of the temperature control block 11, which is configured to convey the cooling air 34 admitted via the inlet opening 25 from the outside to the inside of the cooling air guide body, i.e. into the cooling air path 8, towards the temperature control block 11. For this purpose, the radial fan has blades 29 projecting radially outwards. By means of an electric motor not shown, the fan 27 is set in rotation (about a horizontal axis of rotation 26), at least when a cooling operation is desired, in order to convey cooling air 34 along a flow direction, in particular an inflow direction 35, towards the temperature control block 11.

In particular, the cooling air 34 flows to the temperature control block 11 at an upstream side 37 with the inflow direction 35, flows around the temperature control block 11 laterally and below and leaves the temperature control block 11 at a downstream side 39 opposite the upstream side 37 with an outflow direction 41 which is substantially equal to the inflow direction 35. The vertical direction is designated by reference number 21 and two horizontal directions are designated by reference numbers 19 and 22. Both the inflow direction 35 and the outflow direction 41 are substantially aligned along the horizontal direction 22. Thus, the cooling air of the temperature control block 11 is guided substantially in a horizontally extending flow direction.

The temperature control block 11 has a substantially cylindrical symmetry, with the axis of symmetry 43 shown in FIG. 3A and FIG. 3C. The first fins 17 and the cooling channels 23, which are formed on a shell surface 45 in a side wall 46 of the temperature control block 11, also obey the cylindrical symmetry. Not only the shell surface 45, but also a lower outer surface 47 of the temperature control block 11 are exposed to the cooling air 34 within the cooling air guide body 15. The first fins 17 are each formed in a circular circumference around the temperature control block, and form parts of the shell surface 45 of the temperature control block 11.

As can be seen, for example, from FIGS. 3A, 3B, 3C, the first fins 17 extend parallel to each other in different horizontal planes vertically spaced apart from each other. A first circular cooling channel 23 is formed between each two adjacent first fins 17, within which cooling air 34 flows in the circumferential direction 49 or 51 of the temperature control block 11 in a clockwise direction 51 in one part of the cooling channel and in a counterclockwise direction 49 in another opposite part of the cooling channel.

At the lower surface 47, the temperature control block 11 comprises second fins 18. The second fins 18 extend parallel to each other in a (single) horizontal plane along the horizontal direction 22 and are laterally spaced apart from each other in a horizontal direction 19 perpendicular to the flow direction 35, 41 of the cooling air 34. A second, in particular rectilinear, cooling channel 20 is formed between each two adjacent second fins 18, within which the cooling air 34 flows.

As illustrated in FIGS. 1 and 2, at least one thermal protection element 53 is arranged within the cooling air guide body 15 upstream of the temperature control block 11, which absorbs parts or portions of a thermal radiation 55 originating from the temperature control block 11 and/or reduces a convection of air from the temperature control block 11 to another component arranged upstream. In the illustrated embodiment, the thermal protection element 53 is formed by two pivotable thermal protection flaps 57, wherein the thermal protection flaps 57, in the open state, in particular in a vertical position, substantially clears the cooling air path and, in the closed state, at least partially blocks the air path. The thermal protection flaps are pivotable about horizontally extending axes of rotation 59 and may transition from the closed state (vertical position) 57 illustrated in FIG. 1 to an open state 57′ shown in dashed lines, wherein the flaps may be brought into an almost horizontal orientation. The thermal protection flaps 57 may transition from the closed state 57 to the open state 57′ solely by the flow of cooling air 34 during operation of the fan 27.

The temperature control device 7 illustrated in FIGS. 1 and 2 with the temperature control block 11 illustrated in FIGS. 3A, 3B, 3C is primarily suitable for use in flash point testers employing the Pensky-Martens and/or Cleveland analysis methods as their primary application. Essential components of the temperature control device 7 are the finned heating block 11, which is positioned in a cooling air path 8. The heating block (also referred to as the temperature control block) may be made of, for example, a metallic high temperature resistant metal alloy.

The temperature control block further comprises an electric heating wire 61 for heating the temperature control block, which is arranged in particular inside a lower end wall 48 of the bottom side 47 of the temperature control block 11, in particular circumferentially in the circumferential direction. The heating wire 61 further comprises electrical supply lines 63 connected to a suitable power supply and controlled in particular by a controller 70 (see FIG. 1).

Furthermore, the temperature control block 11 comprises a temperature sensor 65 which is configured to measure the temperature of the temperature control block 11 and which is arranged in particular centrally at a lower end wall 48 of the temperature control block 11. Measuring signals 71 of the temperature sensor 65 are supplied to a controller 70 via electrical conduits 67.

FIG. 1 further illustrates the controller 70, which is configured to control the fan 27 via supply line 74 and/or the heating wire 61 via supply lines 63 in response to a temperature signal 71 generated by the temperature sensor 65 via corresponding control signals 73 and 75, respectively. In this way, a desired temperature control of the temperature control block 11 and thus also of the sample within the container 3 may be achieved.

As can be seen, for example, from FIG. 1, an upper edge 77 of the temperature control block 11 is also located within the cooling air path 8, so that this upper edge 77 and a small portion of the side wall of the container 3 may also be cooled by the cooling air 34. In particular, spacers 79 are provided so that a gap is formed between the upper mounting edge of the cooling air path 8 and the upper edge or top termination 77 of the temperature control block. This gap located in the cooling air path 8 may provide for an optimized cooling of the crucible 3 filled with the sample 5, which is inserted into the temperature control block 11 during a flash point determination measurement, as also illustrated in FIG. 1.

At a downstream side 81, the cooling air guide body 15 is open to discharge exhaust air to the surroundings. In the area of the downstream side, there are ventilation gills 42 which draw cooling air into the ventilation path 41 and mix it with the hot air. The fan 27 or ventilator 27 is installed at the front end of the cooling air path 8 in a heat-decoupled manner. Since the temperature control block becomes hot or may be heated up to 650° C. and the fan 27, which among other things consists of plastic parts, could be damaged, the two metallic thermal protection flaps 57 are installed upstream of the temperature control block 11, The flaps 57 are oriented vertically (position 57) during the heating phases, so that the radial fan 27, which is offset downwardly relative to the heating block, is exposed to minimal heat radiation. During the cooling process after flash point determination, the flaps are positioned substantially horizontally by the air movement to reach the position 57′ so that an unobstructed cooling air flow and thus an optimal cooling of the temperature control block 11 together with the sample container 3 is possible. Moreover, the cooling air path 8 is externally covered with an insulating material in the region of the temperature control block position, so that the heating processes for the flash point determination may be optimally controlled.

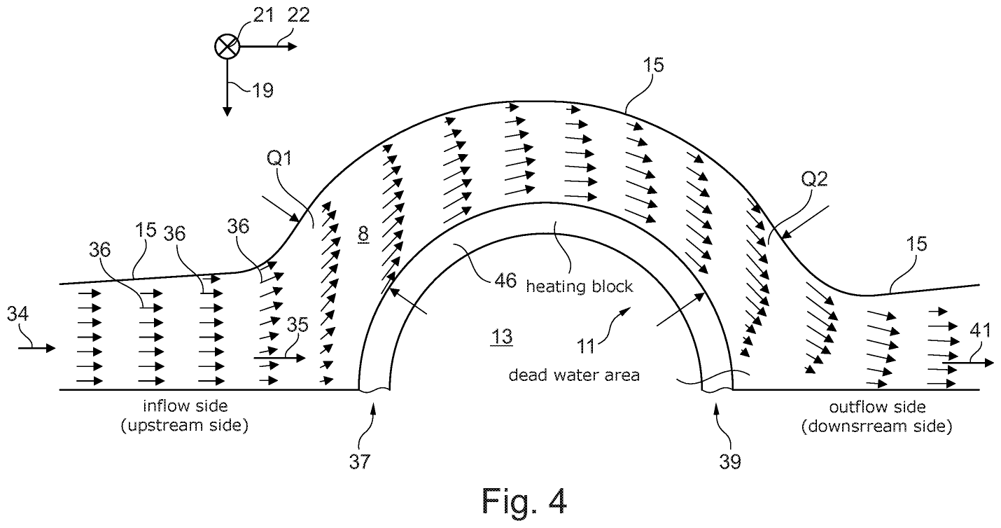

In FIG. 4, the cooling air path 8 within the cooling air guide body 15 is illustrated in a sectional illustration viewed along the vertical direction 21 by an arrow illustration, wherein the direction of the arrows 36 indicates the flow direction and the length of the arrows 36 indicates the flow velocity of the cooling air 34. The cooling air path 8 is delimited by the cooling air guide body 15, and the heating block 11 is arranged within the cooling air path 8.

At the upstream side 37, the cooling air path 8 has a cross-sectional size Q1, while at the downstream side 39, the cooling air path 8 has a cross-sectional size Q2 that is smaller than the cross-sectional size Q1. As a result, the flow velocity in the region of the downstream side 39 is higher than in the region of the upstream side 37. In particular, the cross-sectional size may decrease (continuously or gradually) from the upstream side 37 towards the downstream side 39 in order to result in a continuously or gradually increasing flow velocity.

The following features of the temperature control device promote the cooling process:

1) Circumferential first fins 17 located on the shell surface 45 of the temperature control block 11 provide good heat transfer from the temperature control block 11 to the cooling air 34. At positions of greatest thickness, they comply with the standard and substantially reduce the cooling mass of the temperature control block at positions of least thickness.

2) The fins 17, 18 are aligned along the air flow 35, 41, whereby cooling air 34 flows well around the heating block 11 and as little as possible of the flow is guided over edges transverse to the flow direction. As a result, as few poorly cooling flow separations of the cooling air as possible are formed.

3) In comparison with a heating block without fins, the surface area is multiplied with the fins 17, 18, whereby the heat transfer to the cooling air 34 is increased by approximately the same factor. The circumferential fins 17 of the heating block 11, except for the areas of inflow and outflow, are enclosed by a cylindrical sheet metal part, whereby cooling channels 23 in the form of ring segments are formed on both sides, as also illustrated in FIG. 4. As illustrated in this FIG. 4, the cooling air is directed along a certain path around the heating block by means of these cooling channels and the dead water area is reduced.

4) Due to the cooling, the temperature of the air increases from the upstream or inflow 37 to the downstream or outflow 39, As a result, the temperature gradient to the wall of the heating block is higher on the upstream side than on the downstream side and thus the upstream side of the heating block is cooled better. To reduce this effect, the heating block and cylinder segment of the air channel may be positioned eccentrically so that the annular segment has a higher cross-section Q1 at the upstream side 37 than at the downstream side 39. This increases the flow velocity as the air flows around the heating block 11 and provides better cooling at the downstream side 39 due to the higher flow velocity. The increase in flow velocity is accompanied by pressure loss, therefore a fan should be selected which may offer corresponding pressure ratios (e.g., radial fan).

5) At the bottom side 47 of the heating block there are also fins 18, which are arranged in the flow direction. These additionally support the cooling of the heating block 11 and ensure the cooling of the heating cartridges or the heating wire 61, so as not to delay the cooling process with their residual heat.

Advantages of embodiments of the present invention include a significant mass reduction of the temperature control block due to the provision of the fins, which are formed by varying wall thickness, Due to a reduced temperature control block wall thickness, a reduction in the mass of the temperature control block is achieved, resulting in a higher heating rate and also cooling rate. This results in an efficient and innovative heating/cooling concept conforming to standards for flash point testers and also fire point testers. An improved heating rate during the temperature-controlled processes may be achieved by avoiding air exchange of the heating chamber with the environment by free convection and by minimizing the thermal mass to be heated.

Furthermore, high heating and cooling rates are achieved by the design adaptation of the temperature control block (mass reduction, design of the cooling fins, suitable choice of fan and targeted air guidance), High cooling rates are also achieved by using a radial fan for high air flow per time unit. High cooling rates of the sample container are achieved by recessed mounting of the temperature control block in the cooling air path. The gap of approx. 4.5 mm between the crucible support and the upper edge of the heating block required by the standard is thus in the cooling air flow and additionally supports cooling.

Improved cooling rates and reduction of residual heat of the heating cartridges during the cooling process are achieved. The heating cartridges positioned parallel to the air flow are efficiently cooled by lower cooling fins of the block.

Possible use of commercially available fans made of plastic, despite heating block temperatures of around 650° C., are made possible by a directed offset of the fan downwards relative to the heating block and by fitting protective flaps. The protective flaps are self-opening during the cooling process and do not interfere with the efficiency of the cooling.

1-21. (canceled)

22. A device for tempering a sample located in a container for a flash point determination test and/or fire point determination test, the device comprising:
a temperature control block having a container receptacle for receiving the container;
a cooling air guide body for delimiting a cooling air path in which the temperature control block is arranged;
wherein the temperature control block has an outer surface with fins.

23. The device according to claim 22,
wherein a cooling channel is formed between each two adjacent fins, within which cooling air flows substantially parallel to the fins; and/or
wherein the temperature control block has a wall thickness at positions of fins which is greater than the wall thickness at positions between the fins.

24. The device according to claim 22, wherein the cooling air guided in the cooling air guide body has a substantially horizontally extending flow direction in the region of the temperature control block.

25. The device according to claim 22, wherein the outer surface of the temperature control block comprises a shell surface and a lower outer surface, wherein the shell surface and/or the lower outer surface are exposed to the cooling air within the cooling air guide body.

26. The device according to claim 25, wherein first fins are each formed in a circular circumferential manner and form parts of the shell surface of the temperature control block.

27. The device according to claim 26, wherein the first fins extend parallel to one another in different horizontal planes vertically spaced apart from one another.

28. The device according to claim 26, wherein a first cooling channel is formed between each two adjacent first fins, within which cooling air flows in the circumferential direction of the temperature control block in a clockwise direction in one part of the cooling channel and in an anticlockwise direction in another opposite part of the cooling channel.

29. The device according to claim 26, wherein second fins are provided at the lower outer surface of the temperature control block.

30. The device according to claim 29, wherein the second fins extend parallel to one another in a horizontal plane and are laterally spaced apart from one another in a horizontal direction perpendicular to the flow direction of the cooling air,
wherein a second cooling channel is formed between each two adjacent second fins, within which cooling air flows.

31. The device according to claim 22, wherein at least one thermal protection element is arranged within the cooling air guide body upstream of the temperature control block, said thermal protection element absorbing parts of a thermal radiation originating from the temperature control block and/or reducing a convection of air from the temperature control block to another component.

32. The device according to claim 31, wherein the thermal protection element comprises at least one pivotable thermal protection flap, wherein the thermal protection flap in the open state substantially clears the cooling air path and in the closed state at least partially blocks the cooling air path.

33. The device according to claim 31, wherein the at least one thermal protection flap transitions from the closed state to the open state by pivoting due to a flow of cooling air during a cooling operation.

34. The device according to claim 22, wherein a cross-sectional size of the cooling air path decreases in the region of the temperature control block from upstream to downstream.

35. The device according to claim 22,
wherein the cooling air guide body has an inlet opening for admitting cooling air from outside the device,
wherein the device further comprises a fan upstream of the temperature control block and/or the thermal protection element, which is configured to convey the cooling air admitted via the inlet opening from outside to inside of the cooling air guide body towards the temperature control block.

36. The device according to claim 35, wherein the cooling air guide body is formed,
such that the cooling air flows to the temperature control block on an upstream side with an inflow direction, flows laterally around and/or below the temperature control block and leaves the temperature control block at a downstream side opposite the upstream side with an outflow direction, wherein the outflow direction is substantially equal to the inflow direction.

37. The device according to claim 22, further comprising:
a temperature sensor which is configured to measure the temperature of the temperature control block.

38. The device according to claim 22, wherein the temperature control block comprises an electric heating wire for heating the temperature control block.

39. The device according to claim 37, further comprising:
a controller which is configured to control a fan and/or a heating wire depending on the measured temperature of the temperature control block.

40. A flash point determination apparatus comprising:
a container for receiving a sample to be examined;
a device for tempering the sample located in the container according to claim 22, wherein the container is insertable into the container receptacle of the temperature control block; and
an ignition device for igniting the sample.

41. A method of tempering a sample located in a container for a flash point determination test and/or a fire point determination test, the method comprising:
receiving the container in a container receptacle of a temperature control block;
cooling an outer surface of the temperature control block having fins within a cooling air path delimited by a cooling air guide body.
